(19) United States
(12) Patent Application Publication
(10) Pub. No.: US 2024/0280565 A1
(43) Pub. Date: Aug. 22, 2024

(54) EX VIVO HUMAN MODEL INTENDED FOR EVALUATING ALLERGIC OR PSEUDO-ALLERGIC INFLAMMATORY POTENTIAL

(71) Applicant: GENOSKIN, TOULOUSE (FR)

(72) Inventors: Emeline PAGES, Ramonville Saint Agne (FR); Pascal DESCARGUES, Swampscott, MA (US); Nicolas GAUDENZIO, Tournefeuille (FR)

(21) Appl. No.: 18/562,797

(22) PCT Filed: May 19, 2022
(86) PCT No.: PCT/EP2022/063605

§ 371 Date: Nov. 20, 2023

(30) Foreign Application Priority Data
May 20, 2021 (FR) FR2105293

(51) Int. Cl. G01N 33/50

(52) U.S. Cl. CPC G01N 33/5088

(57) ABSTRACT

This relates to an in vitro method intended to determine the inflammatory potential of a substance including the steps of: ia) administration to a skin explant, topically or by subcutaneous injection, of a composition including the substance; where the skin explant includes the epidermis, the dermis and the skin appendages, as well as a thickness of at least 5 millimeters of hypodermis; ib) determination of the inflammatory response within the skin explant; ic) determination of the level of mast cell degranulation within the skin explant; and ii) determination of the inflammatory potential of the substance and its association or not with an allergic or pseudoallergic potential.

This international application claims the priority of the French patent application FR 21/05293 filed on May 20, 2021, which is incorporated into this application by reference.

### FIELD OF THE INVENTION

The present invention relates to the field of inflammation, whether it be allergic, pseudoallergic, or some other nature, and proposes, more specifically, the first ex vivo human model to determine the inflammatory potential of a compound.

### PRIOR ART

The inflammatory response is an integral part of the immune system's reaction when responding to an attack. However, an inflammatory response can also be induced in a subject following an injection. In addition to any minor inconvenience caused, this inflammatory response can be problematic, particularly if it is an allergic response. Therefore, it is important to be able to predict, prior to any injection, the inflammatory response induced by a substance.

In relation with the allergy, the biological phenomenon resulting in the development of such an allergy generally occurs in two successive phases, with an initial phase known as asymptomatic “sensitization” and a second phase of symptomatic “allergic reaction”.

The sensitization phase begins when the individual comes into contact for the first time with a compound, called the allergen, that generates an allergic response. This compound is then recognized and considered as a foreign substance by certain cells of the immune system, which are present in large quantities in the skin and mucous membranes (antigen-presenting cells, e.g., dendritic cells). These cells then present the allergen on their surface which allows for the production of immunoglobulin E (IgE) by other cells.

These IgEs quickly pass into the blood and attach to cells called mast cells, which are largely present in the skin and mucous membranes (locations where allergens are most likely to penetrate). This process of IgE binding is called “sensitization” because it makes mast cells susceptible to activation upon subsequent encounters with the same antigen. This first phase is silent, that is to say that the subject is asymptomatic in the sensitization phase.

In subsequent contact between the allergen and the “sensitized” organism, the allergen will attach to the IgE present on the surface of the mast cells, causing the latter to activate. This activation results in the degranulation of mast cells which leads to a massive release of histamine and inflammatory mediators in the body.

The mast cell is a white blood cells that is present in connective tissues and is characterized by the presence of numerous secretory granules in its cytoplasm. These secretory granules contain chemical mediators such as serotonin, histamine, tryptase, or heparin. When the mast cell comes into contact with either an allergen presenting its specific IgE or infectious agents, it degranulates and releases its mediators very quickly through the mechanism of exocytosis. This then triggers immediate inflammatory reactions that are sometimes very serious, such as anaphylaxis which can cause hypotension. This same activation induces, in a more delayed manner (a few hours later), the synthesis of numerous cytokines (such as TNF-alpha), chemokines, and lipid mediators.

During allergic reactions, the histamine released exerts its effects by attaching mainly to the H1 receptors present in the nose, where histamine increases edema and obstruction and causes irritation, sneezing, and mucus secretion; in the skin, where histamine causes erythema, edema, and irritation; and in the lungs, where histamine causes bronchoconstriction.

In regard to mast cells, one must also take into account the pseudoallergic potential. This is a mechanism which is not mediated by IgE (unlike an allergic response) but rather by the MRGPRX2 receptor (Mas-related G protein-coupled receptor X2). This mechanism is also dose-dependent and, therefore, these pseudoallergic reactions could be prevented or reduced when administering a substance by reducing the dose or reducing the speed of administration.

Currently, it is especially difficult to predict inflammatory potential in humans and this is particularly the case concerning the allergic or pseudoallergic risk associated with a compound or composition.

Ex vivo models have already been developed, such as that described in patent KR 2021 0034733A, which allows for an in vitro method for determining the inflammatory and allergic potential of a substance. However, in addition to the complexity of implementation, the possibility of extrapolating to humans the results obtained in these models is questionable. In the case of patent KR 2021 0034733A, the claimed method requires the manufacture of a dedicated three-dimensional skin model that integrates a co-culture of fibroblasts from either human skin or rabbit cornea with reporter cell lines (“monitoring cells”) from mouse macrophages (RAW264.7) or rat mast cells (RBL-2H3).

Also, the only method currently available is based on the use of animal models wherein one performs an injection and observes whether or not an inflammatory reaction ensues and, additionally, whether the degranulation of mast cells occurs.

It is easily understood that animal models such as these are less desirable in determining inflammatory potential and, in particular, the allergic or pseudoallergic risk.

Therefore, there is a need to determine easily and quickly, without resorting to animal testing, the association of a compound, composition, or an injectable composition with its inflammatory potential and allergic or pseudoallergic risk in humans.

### SUMMARY OF THE INVENTION

The inventors previously developed an ex vivo model of human skin that makes it possible to test the subcutaneous injection of a solution without requiring animal testing. They have now demonstrated that not only were granulocytes present in said human skin model, but also that these granulocytes retained their function and subsequent capacity for degranulation. This result was all the more unexpected since mast cells were documented to have lost functionality, including significant apoptosis during the first few days (see KIVINEN et al., Experimental Dermatology, vol. 12, pp. 53-60, 2003).

Therefore, this discovery of the inventors makes it possible, following a composition's “subcutaneous” injection or application to the epidermis in this same ex vivo model of human skin, not only to determine the behavior of the granulocytes present and deduce the inflammatory potential of a composition (injected or applied), in particular the existence of an allergic or pseudoallergic reaction, but also to determine the nature of the latter.

Consequently, this allows for the testing of inflammatory potential in humans, in particular the allergic or pseudoallergic potential of an injectable solution, without having to resort to an animal model.

Thus, an initial object of the invention concerns an in vitro method intended to determine the inflammatory potential of a substance and comprises the following steps:
ia) The administration to a skin explant, topically or by subcutaneous injection, of a composition comprising the substance, and where the skin explant comprises the epidermis, the dermis, and the skin appendages, as well as a thickness of at least 5 millimeters of hypodermis;
ib) The determination of the inflammatory response within the skin explant;
ic) The determination of the level of mast cell degranulation within the skin explant; and
ii) The determination of the inflammatory potential of the substance and its association or not with an allergic or pseudoallergic potential.

Preferably, step ia) consists of the subcutaneous injection within the explant of a compound/composition comprising the substance.

According to a preferred embodiment, the method further comprises a step id) of determining the level of degranulation in a culture of mast cells, preferably a culture of primary human mast cells, after incubation thereof in the presence of different concentrations of the substance.

This complementary step, in addition to confirming mast cell degranulation in the substance's presence and allowing for the establishment of an allergic or pseudoallergic potential, enables the determination of the median effective concentration (EC50) corresponding to the necessary concentration of a substance to induce a median degranulation (between the absence of mast cell degranulation to their maximum degranulation).

According to another preferred embodiment, the method further comprises a step ie) of determining the agonist potential of the substance with respect to the MRGPRX2 receptor (Mas-Related G-Protein coupled Receptor member X2).

The method according to the invention makes it possible to distinguish between the allergic or pseudoallergic potential of the substance and, in the case of pseudoallergic potential, to determine the maximum concentration of a substance that can be injected without inducing a pseudoallergic reaction.

### DETAILED DESCRIPTION OF THE INVENTION

The term “substance” denotes a substance of any kind (i.e., protein, carbohydrate, lipid, etc.), which may be of synthetic or natural origin and of which the inflammatory and/or allergic potential one wishes to determine.

The phrase “skin explant” denotes a piece of skin comprising the epidermis, the dermis, and the skin appendages, as well as a thickness of at least 5 mm of the hypodermis (preferably between 5 and 15 mm of hypodermis, and even more preferably, between 5 and 10 mm of hypodermis).

The skin appendages correspond to hair follicles, sebaceous glands, and sweat glands. The hypodermis is the tissue layer located immediately below the dermis of the skin. The hypodermis consists of well-vascularized loose connective tissue and adipose tissue.

If said skin explant is taken from a mammal, one can opt for either humans or pigs. However, given the preferred outcome of the method according to the invention, a human skin explant is preferable.

In connection with the origin of the skin explant, said explant can be obtained via a surgical procedure from any part of the body that includes skin, including the abdomen, chest, buttocks, back, or even, why not, the scalp.

The skin explant is prepared as described in the international application WO/2019/170281.

In detail, said skin explant was submerged, with the exception of the epidermis, in a liquid matrix capable of solidifying like blood plasma and comprising a solution derived from blood plasma (e.g., a dilution of blood plasma in physiological buffer, in particular diluting blood plasma to at least 10%, 20%, 30%, or even at least 40% by weight (weight/matrix total weight)), a fibrinogen solution, a collagen solution, a gelatin solution, synthetic polymer solutions, natural polymer solutions (e.g. agarose (low melting/low melting point agarose or agar), starch, polysaccharides), and mixtures thereof. For more details relating to the matrices capable of solidifying and the methods for placing the skin explants therein, one can consult European patent no. EP 2 882 290 B1.

A liquid matrix capable of solidifying is a liquid solution comprising at least one specific compound/composition whose concentration in said liquid solution is such that, when appropriate conditions are implemented, such as specific temperature conditions in particular, the liquid solution takes on a solid or gel-like consistency. Said compound or specific composition may be of animal, plant, or synthetic origin; its nature and concentration are determined according to the desired physicochemical characteristics of the matrix when it is solidified, in particular the flexibility and strength of the matrix.

Ideally, said liquid matrix which is capable of solidification can be chosen from any liquid solution, preferably nutritional, that is capable of solidifying or gelling under particular conditions that are compatible with the survival and culturing of the skin cells making up said explant.

A matrix such as the one mentioned previously is used in an explant culture method comprising the following steps:
i) Partially submerging a cylindrical skin explant in a liquid matrix chosen from a blood plasma-derived, fibrinogen, collagen, low melting point agar or agarose solution, or a mixture of these solutions-said liquid matrix being contained in said insert—where the epidermal surface of the skin explant remains exposed while the dermis is submerged.
ii) Rapidly inducing the solidification of said matrix surrounding said skin explant.
iii) In vitro or ex vivo culturing or keeping alive said skin explant in said matrix once solidified.

Preferably, said liquid or solidified matrix does not contain any growth factor or serum.

In step i), the cylindrical skin explant is partially submerged in a liquid matrix comprising an initial solution of either a blood plasma-derived, fibrinogen, or collagen solution, and a second solution of low melting point agar or agarose. When the liquid matrix contains a solution of agar or agarose with a low melting point, this solution is previously heated for a duration and at a temperature sufficient to be liquid and to remain liquid at approximately 37° C. for a length of time sufficient for mixing with the first solution in said insert and until the deposition of said skin explant.

Preferably, said second solution is previously heated to its melting point, or slightly higher, preferably between 65° C. and 70° C.

Preferably, the low melting point agar or agarose is an agar or agarose whose maximum gelling temperature is between 24° C. and 28° C. and whose melting temperature is greater than 65.5° C. in 1.5% solution.

Preferably and as an example, but without limitation, this agarose is LMP Agarose (low melting point; GIBCO BRL, LIFE TECHNOLOGIES).

Preferably, said second solution is a solution of low melting point agar or agarose whose concentration is between 1% and 5% (preferably in a physiological solution), more preferably between 2% and 5%, between 3% and 4.5%, between 3.5% and 4.5%, or between 3.8% and 4.2%, or between 3.9% and 4.1%, with 4% being the most preferred concentration.

This second solution in low melting point agar or agarose at said concentration and once heated to its melting point, or slightly above, allows for it to be stored in liquid form for at least 1 hour, though preferably for at least 4, 10, or 16 hours at 37° C.

Preferably, in said liquid matrix comprising said first and said second solution of low melting point agar or agarose, the final concentration of low melting point agarose or agar is between 0.1% and 2%, or even more preferably between 0.2% and 1.8%.

Such a concentration makes it possible to obtain a matrix which, once solidified, not only allows said skin explant to survive and its 3D structure to be preserved but is also solid yet sufficiently flexible enough to be non-brittle and resistant to damage from occasional shocks. The solidification of this liquid matrix takes place after depositing said skin explant in said liquid matrix, where said system thus obtained is then left at a temperature between 37° C. and room temperature, preferably at 20° ° C.

According to one particular embodiment, in said liquid matrix comprising said first solution and said second solution of low melting point agarose or agar, the final concentration of low melting point agarose or agar is between 1% and 2%, preferably between 1.25% and 1.75%, more preferably between 1.4% and 1.6%, with 1.5% being the most preferred concentration.

According to another particular embodiment, in said liquid matrix comprising said first solution and said second solution of low melting point agarose or agar, the final concentration of low melting point agarose or agar is between 0.1% and 2%, preferably between 0.2% and 1.75%, with 0.25% being the most preferred concentration. A concentration such as this makes it possible to obtain a matrix that, once solidified, not only allows for said skin explant to survive with its 3D structure intact but is also sufficiently flexible to be non-brittle and resistant to mechanical effects applied to the explant, for example during an effect mimicking the massaging of the skin for the application of a preparation such as, for example, a cream. The solidification of this liquid matrix takes place after depositing the skin biopsy in said matrix, leaving the system thus obtained at a temperature between 37° C. and room temperature, preferably at 20° C.

Preferably, the volume of said liquid matrix is ⅓ to ⅔ of the total volume of the insert, preferably ⅖ to ⅗ of the total volume, with half of the total volume of the insert being the preferred volume.

In another preferred embodiment, in step a), said liquid matrix capable of solidifying contains between 1 mM and 5 mM Ca+2, preferably between 1.5 mM and 4.5 mM Ca+2. According to a preferred embodiment, in step a) said liquid matrix capable of solidifying contains between 1 mM and 2 mM of Ca+2, preferably between 1.2 mM and 1.4 mM of Ca+2. According to another preferred embodiment, in step a) said liquid matrix capable of solidifying contains between 2 mM and 3 mM Ca+2, preferably between 2.5 mM and 2.8 mM Ca+2, and more preferably 2.8 mM Ca+2.

Also preferably, said liquid matrix capable of solidifying contains between 5 and 500 mg/mL of ascorbic acid, preferably between 25 and 75 mg/mL, with 50 mg/ml of ascorbic acid being the most preferred concentration.

More preferably, said liquid matrix capable of solidifying is a medium containing between 1 mM and 5 mM of Ca+2 and between 5 and 500 mg/mL of ascorbic acid.

In a preferred embodiment, said liquid matrix capable of solidifying and contained within the insert in step a) is a mixture of which the first solution in the mixture (the second solution being the agar or agarose solution) is preferably nutritious and also able to solidify due to an increase or decrease in temperature and/or by the addition of a specific compound or composition.

Preferably said liquid matrix capable of solidifying does not contain any growth factor or animal or human serum.

Preferably, in step a), said liquid matrix capable of solidifying does not cover the upper surface of the epidermis before this matrix is solidified in step b).

According to another equally preferred embodiment, in step a) of said culture method, said skin explant is deposited in a liquid matrix capable of solidifying, in particular as indicated above, and of which said liquid matrix is selected from any liquid solution that provides all the nutrients and/or ingredients necessary for its culture, in particular for the maintenance of the initial physiological state of the cells which constitute said explant. Said solution is capable of solidifying or gelling under specific conditions that are compatible with the survival and culture of the skin explant.

Preferably, said liquid matrix capable of solidifying is a liquid solution derived from blood plasma (that has been treated with a reversible anticoagulant) that has been mixed with an agar or agarose solution.

Preferably, said liquid matrix capable of solidifying contains blood plasma, fibrinogen, or collagen mixed with a solution of low melting point agarose or agar

According to an equally preferred embodiment of the culture method, in step a), said liquid matrix capable of solidifying consists of the following: a blood plasma-derived solution containing 25% to 60% v/v, preferably between 35% and 45% v/v, of blood plasma; 70% to 35% of a physiological solution, such as a 0.9% NaCl solution; 5% to 12%, though preferably 8%, of a 1% CaCl2 saline solution; a sufficient concentration of an antifibrinolytic agent to obtain the desired antifibrinolytic activity, preferably between 5% and 2%, and preferably with the antifibrinolytic agent being selected from either tranexamic acid or aprotinin; and a solution with between 0.5% and 4%, preferably between 1% and 2%, of low melting point agarose.

According to an equally preferred embodiment of the culture method, in step a), said liquid matrix capable of solidifying is a liquid solution of fibrinogen and thrombin, or of collagen or blood plasma, mixed with a gelatin solution comprising synthetic and/or natural polymeric gels such as agarose gels (in particular agarose or agar gels with low melting points), starch, or polysaccharide gels, and for which incubation at 37° C. allows said solution to solidify.

According to an equally preferred embodiment of the culture method, in step a), said liquid matrix capable of solidifying contains a liquid solution derived from blood plasma that has been treated with an anticoagulant with reversible properties, preferably citrate sodium, and the solidification of said matrix in step b) for this solution can occur in the presence of calcium ions, preferably also in the presence of thrombin.

When the liquid matrix capable of solidifying contains a solution of blood plasma, fibrinogen, or collagen, the solidification of said matrix in step b) can be induced for this solution by adding thrombin, increasing the temperature, or using factors secreted by cells introduced to the matrix, such as primary fibroblasts.

According to an equally preferred embodiment of the explant culture method, in step b), said liquid matrix capable of solidifying is solidified after a maximum of 8 hours, preferably less than 2 hours or less than 1 hour, with a duration of less than 30 min though less than 10 min is the most preferred duration for initiating the solidification phase of the liquid matrix after depositing the skin biopsy in said liquid matrix in step a).

To return to the method according to the invention, and in the case of a skin explant of cylindrical shape, the skin explant will have a diameter of between 10 mm and 50 mm, preferably between 15 mm and 40 mm.

Advantageously, the skin explant would be positioned within an insert that may take multiple forms and in particular corresponds to a suspended or standing insert. Currently, a suspended insert is preferable. The bottom of this insert consists of a porous membrane whose diameter is between 5 mm and 40 mm, preferably between 9.5 mm and 30 mm. As for the porosity of this membrane, it must prevent the liquid matrix from passing through it before said matrix solidifies. Typically, this porous membrane will have a porosity between 0.4 μm and 8 μm, preferably between 0.4 μm and 1.5 μm, with 0.8 μm to 1.2 μm as the most preferred porosity. In terms of material, one can therefore choose a porous membrane from polyethylene terephthalate (PET), nitrocellulose, and polycarbonate membranes. Finally, such inserts that can be cited as an example include those supplied by the companies NUNC, CORNING, BECTON DICKINSON (BD FALCON), MILLIPORE (MILLICELL) which may take the form of inserts with a polycarbonate, PET, or nitrocellulose membrane, and which are prepackaged in multi-well plates of 6, 8, 12, or 24-well culture plates, and whose membrane porosity can vary from 0.4 μm to 8 μm.

The phrase “topical application” denotes an application on the skin explant's epidermis of the composition/compound to be tested.

The phrase “subcutaneous injection” denotes an injection which is carried out in the hypodermis of the skin explant, which also qualifies it as a “hypodermic” injection. This type of injection, which is well known to those skilled in the art, generally requires pinching the skin with fingers to create a skin fold, then the subcutaneous injection is carried out in said skin fold.

According to a preferred embodiment, step ia) consists of the subcutaneous injection of a composition comprising the substance into a skin explant.

The composition in question is a composition to be tested which is in liquid form. Advantageously, the volume of said composition would be between 10 μl and 1 ml, preferably between 10 μl and 500 μl and, more preferably, be between 10 μl and 200 μl.

The needle for injecting the composition typically has sufficient length to reach the hypodermis. Thus, it is preferable to use needles having a length greater than or equal to 10 mm. As an example of such needles, needles having a length of 12, 16, 20, 25, 30, 35, 40, or even 45 mm could be used. Advantageously therefore, the needle would have a length of between 16 mm and 45 mm, preferably a length of 20 mm to 40 mm. As for the diameter of the needle to be used, it can simply be identified by those skilled in the art based on their general knowledge. Typically, such hypodermic needles are of the following sizes: 18 G, 19 G, 20 G, 21 G, 22 G, 23 G, 25 G, 26 G, 27 G, 28 G, 29 G, 30 G, or even 31 G.

This injection step can be carried out by a researcher, who pinches the skin so as to allow for the formation of a skin fold and thus facilitate the subcutaneous injection. Currently, this injection step can also be carried out by an automatic injection device. Typically, said device allows for an injection at a predetermined depth, relative to the surface of the epidermis, so as to obtain a subcutaneous injection.

Step ib) of determining the inflammatory response within the skin explant is carried out by monitoring inflammatory markers which are well known to those skilled in the art. Such markers of inflammation may be present within the skin explant, the matrix in which the skin explant is partially submerged, and/or in the culture medium. Markers of inflammation that can be cited, but are not limited to, include cytokines and antibacterial proteins. Currently, it is possible to cite proteins involved in lipid biosynthesis or any other molecule whose expression varies between an inflamed state and a non-inflamed state (see in particular SERHAN & WARD, Molecular and Cellular Basis of Inflammation, Humana Press). It is also possible to analyze the monitoring of differentially expressed markers from transcriptomic, proteomic, or lipidomic studies.

Cytokines that can be cited as markers of inflammation include interleukins and their receptors. Citable examples of interleukins include IL-1A, IL-1B, IL-5, IL-6, IL-7, IL-8, IL-9, IL-10, IL-12, IL-17A, IL-17C, IL-17F, IL-19, IL-21, IL-22, IL-23, IL-27, IL-31 and IL-33, whereas examples of interleukin receptors include IL10RA, IL10RB, IL1R1, IL5RA (CD125) and IL9R.

As cytokines that can be used as markers of inflammation, chemokines—chemotactic cytokines—can also be cited that control the migration patterns and the positioning of immune cells, as well as their receptors. As examples of cytokines, we can cite C5, Eotaxin, MCP-4, TARC, MCP-1, MIP-3A, CCL22, CCL23, MIP-1B, RANTES, MCP-3, MCP-2, CX3CL1, IL8RA, INP10, L8RB and CXCL3. Concerning chemokine receptors, we can cite CCL13 (MCP-4), CCR1, CCR2, CCR3, CCR4, CCR5, CCR6, CCR8, CX3CR1, CXCR1, and CXCR2.

It should be noted that other cytokines (than interleukins and chemokines) can be considered as markers of inflammation. As examples of such cytokines, one can cite MCP-1, GM-CSF, TNFSF5, M-CSF, G-CSF, TNFSF6, IFNA2, IFNG, TNFA, TNFB, MIF, NAMPT, TRAIL, and IFNA1.

Among the antibacterial proteins, one can cite antimicrobial peptides (AMPs), also called host defense peptides, which are part of the innate immune response and have a length of less than 50 amino acids. Unlike the majority of conventional antibiotics, it appears that antimicrobial peptides frequently destabilize biological membranes, can form transmembrane pores, and can also strengthen the immune response by acting as immunomodulators. As antimicrobial peptides, it is notably worth citing defensins (e.g., beta-defensins and cathelicidins such as LL-37). There are also other antibacterial proteins besides antimicrobial peptides, for example the S100 proteins (S100A7 (psoriasin) and S100A15 (koebnerisin)).

Typically, markers of inflammation are chosen from molecules secreted by structural cells (e.g., keratinocytes, stromal cells), adipocytes, and immune system cells (e.g., macrophages, dendritic cells, T-lymphocytes, mast cells). Citable examples of such markers of inflammation include M-CSF, G-CSF, TNFSF6, IFNA2, IFNG, RANTES, MCP-3, MCP-2, CX3CL1, TNFA, TNFB, MIF, NAMPT, TRAIL and IFNA1, TNFA, MCP1, VEGF, IP-10, MDC, MIP-1B, IL-17A, IL-17C, IL-17F, TNFB, IL-27, MCP-4, MIP-1A, IL-22, IL-1B, IL-12/IL-23p40, GM-CSF, IFNG, IL-12p70, IL-23, IL-31, EOTAXIN, IL-6, IL-4, IL-13, IL-5, IL-8, IL-15, BETA-HEXOSAMINIDASE, HISTAMINE, TRYPTASE, and CHYMASE.

Preferably, markers of inflammation present in the culture medium will be chosen. Citable examples of such markers include TNFA, MCP-1, EOTAXIN-3, VEGF, IP-10, IL-2, MIP-3A, MIP-1B, IL-17A, MIP-1A, IL-22, IL-1B, IL-12/IL-23p40, GM-CSF, IFNG, IL-12p70, IL-23, EOTAXIN, IL-6, IL-8, BETA-HEXOSAMINIDASE, HISTAMINE, and TRYPTASE.

Step ic) of determining the level of mast cell degranulation can be carried out using techniques well known to those skilled in the art. Techniques that can be used for this step include ELISA or other colorimetric techniques to measure the presence of inflammatory mediators contained in mast cell granules, such as histamine or tryptase, or secreted de novo such as lipid mediators or cytokines/chemokines, or even fluorescence, immunofluorescence, or fluorochrome techniques specific to mast cell granules.

Advantageously, this step ic) would be carried out within a maximum of 6 hours following the administration step ia), preferably within a maximum of 4 hours.

The inventors have in fact shown that, if the granulocytes functioned properly, the monitoring of degranulation was optimal during this interval. Of note, the inventors also demonstrated that it was not possible to subsequently induce further mast cell degranulation.

Preferably, a single step ia) of administration of a composition/compound comprising the substance is carried out per skin explant.

According to a preferred embodiment, step ic) of determining the level of mast cell degranulation within the skin explant is carried out by fluorescence analysis.

Advantageously, this step ic) of determining the level of mast cell degranulation uses avidin.

Avidin is in fact a glycoprotein which binds very specifically to the heparin contained in the granules of mast cells (THARP et al., J. Histochem. Cytochem., vol. 33, p:27-32, 1985). Thus, during the degranulation process once the granules are externalized they become directly accessible to avidin. Now, and in the case of tissue fixation, the intracellular granules become accessible to avidin as soon as tissue permeabilization is carried out. This avidin can be conjugated to a fluorochrome (avidin-FITC, avidin-Alexa Fluor™ 488, avidin-sulforhodamine 101 or any other fluorescent molecule) or with a bioluminescent molecule. Currently, it is also possible to use avidin alone (unconjugated) and in combination with a molecule that is complementary to it. The following examples of such a molecule may be cited: biotin conjugated to a fluorochrome, a bioluminescent molecule, or any other identifiable molecule.

Currently, the determination of the extent of mast cell degranulation can use other markers, in particular the nucleus or the plasma membrane, so as to facilitate the identification of granules externalized from mast cells. It is also possible to measure tryptase, histamine, beta-hexosaminidase, chymase, or any other molecules preformed within the mast cell granules and released into the culture medium during degranulation.

Typically, the determination of the level of mast cell degranulation within the skin explant can be carried out by following the protocol described in GAUDENZIO et al. (J. Clin. Invest., vol. 126, p:3981-3998, 2016)

Preferably, step ic) of determining the level of mast cell degranulation will be carried out on at least one histological section made from the skin explant.

To do this, one may use the well-known methods of immunohistochemistry that utilize the fixation of the skin explant, the embedding of the explant (e.g., paraffin, OCT, Epon), before its conservation, and finally the production of histological sections from the embedded block. The details of such methods are described for example in Immunohistochemistry: Basics and Methods by Igor BUCHWALOW (Editions SPRINGER).

The usable histological sections may have a very significant thickness of up to 500 μm. The histological section will thus have a thickness of between 1 μm and 500 μm. Currently, it is possible to use sections with more classic dimensions with a thickness between 2 and 25 μm.

As for determining the level of mast cell degranulation within the explant itself, one first determines, for each identified mast cell, whether it is associated with low, moderate, or high degranulation, then determines the proportion (percentage) of mast cells associated with each of these levels of degranulation (low, moderate, and high). The level of degranulation can also be analyzed in an automated manner via image analysis software, computer algorithm, or artificial intelligence techniques such as machine-learning or deep-learning.

In detail, a mast cell with a low level of degranulation corresponds to a mast cell with 0 to 2 granules around it (or to a cell with a smooth outline); a mast cell with a moderate level of degranulation corresponds to a mast cell with 3 to 6 granules around it (or to a cell with a granular appearance); and a mast cell with a high level of degranulation corresponds to a mast cell with more than 6 granules around it (or to a cell with an exposed shape).

For control purposes, the same steps ia), ib) and ic) may be carried out with a negative control (e.g. PBS) and/or with a composition comprising a positive control corresponding to a substance known to induce inflammation or, more specifically mast cell degranulation, such as for example IgE/antigen, IgE/anti-IgE complexes, compound 48/80, substance P, various secretagogues, products from pathogens, or therapeutic molecules known to induce reactions at the injection site (e.g., anakinra, cetrorelix or icatibant).

Step ii) of determining the inflammatory potential, and more specifically the allergic or pseudoallergic potential of the substance, can then simply be carried out with regard to the result of steps ib) and ic).

Thus, it will be possible to determine whether the substance presents an inflammatory potential with regard to an increase in inflammatory markers at the end of step ib).

It will then be possible to determine whether or not this inflammatory potential is associated with an allergic or pseudoallergic potential at the end of step ic) with regard to the proportions of mast cells undergoing weak, moderate, or strong degranulation at end of this step.

Thus, a substance associated with a proportion of more than 50% of granulocytes presenting a low level of degranulation and/or of less than 10% a high level of degranulation has a low, or even zero, allergic or pseudoallergic potential.

The inventors have also shown that, in the explant in culture, there is a basic level of mast cell degranulation comparable to that observed in vivo in animals, which was not obvious and supports the interest of this model.

Conversely, a substance associated with a proportion of granulocytes of more than 50% presenting a high level of degranulation indicates a high allergic or pseudoallergic potential.

Here again, the inventors have demonstrated that this level of degranulation observed in the cultured explant is comparable to that observed in vivo in animals when an inducer of degranulation was administered. It should also be noted that, unlike the situation observed with the basic level of degranulation, an inhomogeneous spatial distribution of mast cells undergoing degranulation was observed. The proportion of mast cells undergoing strong degranulation increases according to proximity to the injection site. This result once again confirms the relevance of the model according to the invention.

Finally, a substance associated with an intermediate proportion of granulocytes not exceeding either of the two previous thresholds, has an intermediate or moderate allergic or pseudoallergic potential.

According to a preferred embodiment, the method further comprises a step id) of determining the level of degranulation in a mast cell culture after incubation thereof in the presence of different concentrations of the substance.

Step ii) of determining the inflammatory potential of the substance then makes it possible, in conjunction with this step id) and in addition to confirming degranulation by mast cells in the presence of the substance, to determine the median effective concentration (EC50) of the substance for the induction of mast cell degranulation.

Typically, a culture of primary mast cells, preferably human, will be used.

Such a culture of mast cells can be obtained by methods well known to those skilled in the art. As an example, the protocol described in GAUDENZIO et al. (J. Allergy Clin. Immunol., vol. 131(5), p: 1400-7, 2013) and in GAUDENZIO et al. (previously cited, 2016) can be used and are described in the Examples. To do this, mast cells are derived in appropriate culture media from hematopoietic progenitors (e.g., CD34+, CD133+ cells) present in the circulating blood of healthy donors (e.g., adult peripheral blood, umbilical cord blood).

The determination of mast cell degranulation in culture can then be performed using techniques well known to those skilled in the art, which have been mentioned previously. Typically, an assay of mast cell markers is performed before and after stimulation of the explant (e.g., approximately 1 hour). Mast cell markers preformed in secretory granules that can be cited include beta-hexosaminidase, tryptase, chymase, or even histamine. Now, it is also possible to measure the secretion of cytokines and chemokines including M-CSF, G-CSF, TNFSF6, IFNA2, IFNG, RANTES, MCP-3, MCP-2, CX3CL1, TNFA, TNFB, MIF, NAMPT, TRAIL and IFNA1; TNFA, MCP1, VEGF, IP-10, MDC, MIP-1B, IL-17A, IL-17C, IL-17F, TNFB, IL-27, MCP-4, MIP-1A, IL-22, IL-1B, IL-12/IL-23p40, GM-CSF, IFNG, IL-12p70, IL-23, IL-31, EOTAXIN, IL-6, IL-4, IL-13, IL-5, IL-8 and IL-15. The identification of these granulocyte markers can be carried out by colorimetric tests or ELISA type tests in 96 or 384 well plates. In addition, flow cytometry allows for the detection of the increase in exocytosis markers on the surface of mast cells such as lamp-1, annexin 5, or by labeling with fluorescent avidin or others.

A percentage of degranulation is defined in relation to a positive control, i.e., the lysis of all mast cells in a well via a detergent (e.g., TRITON X100 used at 1%). Concerning definitions of allergic or pseudoallergic potential, a molecule has a low allergic or pseudoallergic potential if the percentage of mast cell degranulation is between 5%, and 15%, a moderate potential if the percentage of mast cell degranulation is between 15% and 30%, and a strong potential if the percentage of mast cell degranulation is greater than 30%.

According to another preferred embodiment, the method further comprises a step ie) of determining the agonist potential of the substance with respect to the MRGPRX2 receptor.

Such a step can be carried out simply by those skilled in the art and is described in the examples. To do this, cells are transformed so as to express the human MRGPRX2 receptor (see for example accession numbers Q96LB1, NP_001290544.1, NP_473371.1, ACG60653.1, EAW68359.1, or even AAH63450.1). Activation of the MRGPRX2 receptor results in a decrease in the intracellular concentration of calcium ions, which can be measured in order to extrapolate the activity of the MRGPRX2 receptor and, in the presence of the substance, the agonist potential of said substance with respect to the MRGPRX2 receptor. Said measurement of receptor activation can also be performed via bioluminescence (e.g., Fluo-4), whereby, if an increase in the fluorescent signal is detected after the addition of the stimulating molecule, then the molecule can be considered as an agonist of the MRGPRX2 receptor.

In light of the results obtained in connection with steps ib), ic), and id), this step ie) makes it possible to know whether the mast cell degranulation reveals an allergic potential or a pseudoallergic potential. Indeed, MRGPRX2 is the receptor responsible for pseudoallergies. Therefore, these pseudoallergic reactions could be prevented, in the event of new administration of the substance, by reducing either the dose or speed of administration, or co-injection of an MRGPRX2 receptor antagonist.

Also, step ii) of determining the inflammatory potential of the substance then makes it possible to determine whether or not the mast cell degranulation induced by the substance results from a pseudoallergic reaction. Once a substance is identified as having pseudoallergic potential, step ii) therefore makes it possible to identify the substances that should, for example, be combined with at least one mast cell stabilizer in order to facilitate their injection.

Mast cell stabilizers are medications used to prevent or control certain allergic disorders that are well known to those skilled in the art. These compounds block mast cell degranulation, stabilizing the cell and thereby preventing the release of histamine and associated mediators. As examples of mast cell stabilizers, mention may be made of loratadine, desloratadine, sodium cromoglycate, ketotifen, olopatadine, rupatadine, mepolizumab, omalizumab, pemirolast, nedocromil, azelastine, β2-agonists, quercetin, luteolin, rutin, and even vitamin D.

Therefore, and in the case of a pseudoallergic reaction, the results obtained at the end of step id) also make it possible to determine the concentration of substance to be used so as not to induce mast cell degranulation or low degranulation.

The following examples are given solely by way of illustration of the subject of the present invention and of which they do not constitute a limitation in any way.

### EXAMPLES

### 1—Preparation of Skin Explants

Skin explants are prepared from complete skin samples from different donors and include the epidermis, dermis, and hypodermis (1.5 cm to 2 cm). The explants (epidermis, dermis, and hypodermis) are then cut using a circular metal punch to obtain cylinders of 11 mm to 20 mm in diameter, where the thickness of the hypodermis is adjusted to the desired value (0.5 cm to 1 cm). Finally, these explants were kept floating in a buffered saline solution until being partially submerged in the liquid matrix. Said submerging was carried out in accordance with a method similar to that used for the NATIVESKIN™ model. Briefly, the skin explant is delicately placed into an insert (8-well MILLICELL™ chamber slide) with a porous membrane at the bottom (in PET, porosity 1 μm) and containing a blood plasma-derived solution that has been treated with an anticoagulant that undergoes reversal in the presence of calcium ions (sodium citrate). This solution consists of 42% of blood plasma, 50% of 0.9% NaCl solution, 8% of 1% CaCl2 saline solution, an antifibrinolytic agent (tranexamic acid or aprotinin), and low melting point agarose at 0.7% (LMP Agarose, GIBCOBRL, LIFE TECHNOLOGIES) (melted in an incubator at 65.5° C.). The antifibrinolytic agent functions by inhibiting enzymes secreted by the skin explant that are likely to degrade the plasma matrix, thereby thus maintaining the integrity of the explant.

### 2—Mast Cells and Skin Explant

2.1—Identification of Mast Cells within the Skin Explant

The presence of mast cells within the explant was analyzed by avidin labeling via an anti-tryptase antibody. The mast cells were found in identical numbers between the first and the 5th day of culture. Following the injection of a proinflammatory compound (e.g., a vaccine formulation), an increase in the secretion of proinflammatory cytokines was detected in the culture medium.

2.2—Demonstration of Mast Cell Degranulation Activity within the Skin Explant

Following subcutaneous injection of 100 μL of a 50 μg/mL solution of either compound 48/80 (an MRGPRX2 agonist) or control water into the skin explant, mast cell degranulation was quantified at 1-, 4-, 6-, and 24-hours post-injection. The best signal compared to the control was observed between 0- and 4-hours post-injection. It was observed that from 6-hours onwards there was spontaneous degranulation of mast cells even in the absence of stimulation, which could affect subsequent analysis. These results therefore demonstrate that the skin explant includes mast cells that are maintained over time and are capable of degranulation. Consequently, the skin explant allows for the determination of the allergic or pseudoallergic potential of a substance without having to resort to an animal model.

### 3—Determination of the Inflammatory Potential of a Substance (e.g., Cetrotide or Cetrorelix)

### 3.1—Inflammatory Potential

A CETROTIDE solution of 100 μL with a cetrorelix concentration of 0.25 mg/l mL (luteinizing hormone inhibitor that competes by binding to its receptor) was injected into the adipose tissue of an explant, as described previously, using a syringe and a 12 mm long, 27 G needle. As a negative and positive control, 100 μL of a PBS solution and 100 μL of a concentrated solution of compound 48/80 (50 μg/mL, an MRGPRX2 agonist), respectively, were each injected into the adipose tissue of different explants, as described previously.

The culture medium or matrix supporting the explants are collected and an assay of cytokines is conducted using an ELISA-type methods or any other type of method intended to detect and quantify the presence of cytokines.

The results confirmed the inflammatory potential of cetrorelix.

### 3.2—Allergic or Pseudoallergic Potential of the Substance

A CETROTIDE solution of 100 μL with a cetrorelix concentration of 0.25 mg/l mL (luteinizing hormone inhibitor that competes by binding to its receptor) was injected into the adipose tissue of an explant, as described previously, using a syringe and a 12 mm long, 27 G needle. As a negative and positive control, 100 μL of a PBS solution and 100 μL of a concentrated solution of compound 48/80 (50 μg/mL, an MRGPRX2 agonist), respectively, were each injected into the adipose tissue of different explants, as described previously.

Said explants were then cultured (incubator at 37° C., 5% CO2, and high saturation humidity) for 1 to 4 hours before fixation (4% paraformaldehyde solution) and embedding in a paraffin block in order to perform a histological analysis.

In detail, the skin explants were dehydrated first by an alcohol bath and then by xylene bath. Finally, a paraffin bath allowed for the water previously contained in the skin explant to be replaced with paraffin The paraffin-impregnated samples are removed from their bath and transferred to a container whose bottom is lined with absorbent paper, in order to be brought near to the embedding station. The samples, enclosed in histology cassettes, are immersed in liquid paraffin at 56° C., which remelts the paraffin inside the samples. For each sample, the histology cassette is opened; optionally, the sample may be cut into two. Next, an embedding mold is filled with liquid paraffin and the sample (or 2 sample pieces) is placed in the mold and oriented in the desired direction for sectioning. At that moment the mold is transferred to a refrigerated support in order to solidify the paraffin at the bottom of the mold and to maintain the sample. The cover of the histology cassette upon which the sample reference appears is placed on top of the mold in such a way that the paraffin covers it (possible to add paraffin if necessary), then the entire mold is placed in a cold environment (refrigerator, freezer, cold room, etc.) for several minutes (5 to 6) to solidify the paraffin into a block, effectively trapping the sample in the correct orientation alongside the histology cassette cover, which then becomes the support for the block. Once the paraffin block has completely solidified, it is removed from the mold. It is now possible to scrape off excess paraffin with a spatula along the sides of the cassette's cover. Serial sections with a thickness varying from 4 to 5 μm are then made over the entire length of the paraffin block containing the sample.

In order to determine the level of mast cell degranulation within the tissue under the different conditions, labeling is carried out via fluorochrome-conjugated avidin, thereby permitting the detection of mast cell granules. The following step allows the fixed and paraffin-impregnated sections to be deparaffinized and rehydrated. The sections are incubated for 30 minutes at room temperature in Citrate pH6 buffer, then saturated and permeabilized for 40 minutes at 37° C. with a solution of goat serum and 0.1% Triton. The sections are then incubated for one hour at room temperature in a humid chamber with 5 μg/mL Avidin-Sulforhodamine 101 (Avidin TEXAS RED, MERCK). Staining of the cell nuclei is then carried out by incubating the sections with DAPI (D9542, SIGMA) at 1/1000 for 3 minutes at room temperature. Next, mounting medium is added and a coverslip is placed on the sections. The slides are then analyzed under a fluorescence microscope to determine the level of mast cell degranulation in the different explants.

The results of these experiments are presented in Table 1.

TABLE 1

| | Compound Administered | | |
|---|---|---|---|
| Percentage of granulocytes with | PBS | compound 48/80 | Cetrotide |
| Low level of degranulation | 67.0 | 36.1 | 12.5 |
| Moderate level of degranulation | 28.0 | 37.1 | 31.6 |
| High level of degranulation | 5.0 | 26.8 | 55.9 |
| Allergic or pseudoallergic potential | Low | Moderate | High |

Thus, these results permitted the determination of both the level of mast cell degranulation identified in the samples and, after integration, the inflammatory potential of each injected substance. In this case, the results confirm that the inflammatory potential of cetrotide is associated with mast cell degranulation. Consequently, cetrotide has a high allergic or pseudoallergic potential.

### 3.3—Confirmation of the Allergic or Pseudoallergic Potential of the Substance

A primary human mast cell culture was obtained according to the protocol described in GAUDENZIO et al. (J. Allergy Clin. Immunol., vol. 131(5), p: 1400-7, 2013) and in GAUDENZIO et al. (previously cited, 2016). Briefly, PBMCs from blood samples from different donors were isolated via Ficoll density gradient centrifugation. Then hematopoietic progenitors expressing the CD34+ marker were isolated from among the PBMCs using magnetic cell separation (possibly also achievable by FACS). Next, these cells were cultured towards mast cell maturation by cultivation in a serum-free culture medium supplemented in particular with IL-6, IL-3, and recombinant SCF for a period of approximately 3 month.

Mast cells are considered mature and ready for use upon exhibiting both cytoplasmic granules, as labeled by fluorochrome-conjugated avidin, and the capacity for degranulation in response to stimulation by IgE/antigen complexes (IgE/anti-IgE) or by MRGPRX2 receptor agonists (e.g., compound 48/80 or substance P).

The determination of mast cell degranulation in culture was then performed using an assay of granulocyte markers (e.g., beta-hexosaminidase) before and after stimulation (approximately 1 hour) with substance P, compound 48/80, and IgE/anti-IgE complexes. This assay was carried out via colorimetric testing in 96-well plates.

The results confirm those obtained with the skin explant, namely that cetrotide has a high allergic or pseudoallergic potential.

### 3.4—Allergic or Pseudoallergic Potential of Cetrotide

HEK 293 cells were transformed to express human MRGPRX2 receptors (Accession number Q96LB1).

Activation of the MRGPRX2 receptor results in a decrease in the intracellular concentration of calcium ions, thus the monitoring of this concentration allows for extrapolation of MRGPRX2 receptor activity. To do this, a fluorescent calcium indicator was used, namely Fluo-4.

The transformed cells were placed on a microscope slide equipped with one or more culture wells. The post-acquisition analysis of fluorescence per cell was carried out using standard image processing software (e.g., IMAGEJ).

The cells labeled with Fluo-4 were then incubated in the presence of cetrotide. An analysis of the fluorescence was then made by video recording on a fluorescence microscope over a period of approximately 300 seconds after the incubation began. First, a 20-second sequence is carried out to establish the base line before the addition of the molecule to be tested. Then the molecule to be tested is added for a period of 100 seconds, followed by 100 μM of substance P in order to locate the cells expressing MRGPRX2. Finally, 100 seconds later, 100 UM of ionomycin is added as a positive control for visualizing calcium flow.

The results showed that, after addition of cetrotide, an increase in fluorescent signal was detected in the same cells that responded to substance P and, therefore, were those transfected to express MRGPRX2. Accordingly, cetrotide can be considered an agonist of the MRGPRX2 receptor.

Consequently, the results demonstrated that cetrotide has a strong pseudoallergic potential.

1. An in vitro method intended to determine the inflammatory potential of a substance comprising the steps of:
ia) administering to a skin explant, topically or by subcutaneous injection, of a composition comprising the substance; where said skin explant comprises the epidermis, the dermis, and the skin appendages, as well as a thickness of at least 5 millimeters of the hypodermis.
ib) determining the inflammatory response within the skin explant.
ic) determining the level of mast cell degranulation of within the skin explant.
ii) determining the inflammatory potential of the substance and its association or not with an allergic or pseudoallergic potential.

2. The method according to claim 1, wherein step ia) consists of the subcutaneous injection of a composition comprising the substance.

3. The method according to claim 1,
wherein step ib) of determining the inflammatory response within the skin explant is carried out by monitoring markers of inflammation chosen from the group comprising M-CSF, G-CSF, TNFSF6, IFNA2, IFNG, RANTES, MCP-3, MCP-2, CX3CL1, TNFA, TNFB, MIF, NAMPT, TRAIL and IFNA1, TNFA, MCP1, VEGF, IP-10, MDC, MIP-1B, IL-17A, IL-17C, IL-17F, TNFB, IL-27, MCP-4, MIP-1A, IL-22, IL-1B, IL-12/IL-23p40, GM-CSF, IFNG, IL-12p70, IL-23, IL-31, EOTAXIN, IL-6, IL-4, IL-13, IL-5, IL-8, IL-15, BETA-HEXOSAMINIDASE, HISTAMINE, TRYPTASE, and CHYMASE.

4. The method according to claim 1, wherein step ic) of determining the level of mast cell degranulation within the skin explant utilizes avidin.

5. The method according to claim 1, wherein step ic) of determining the level of degranulation of the mast cells within the skin explant is carried out within a maximum period of 6 hours following the step ia) of administration.

6. The method according to claim 1, wherein step ii) of determining the inflammatory potential of the substance, and more specifically its allergic or pseudoallergic potential, is performed with regard to the proportions of mast cells undergoing low, moderate, or high levels of degranulation at the end of step ia), with:
a substance associated with a proportion of more than 50% of granulocytes exhibiting a low level of degranulation and/or less than 10% exhibiting a high level of degranulation presents a low, or even zero, allergic or pseudoallergic potential.
a substance associated with a proportion of more than 50% of granulocytes exhibiting a high level of degranulation presents a high allergic or pseudoallergic potential.

7. The method according to claim 1, further comprising a step id) of determining the level of degranulation in a culture of mast cells after incubation thereof in the presence of different concentrations of the substance.

8. The method according to claim 7, wherein step ii) of determining the inflammatory potential of the substance also makes it possible to determine the median effective concentration (EC50) of the substance for the induction of mast cell degranulation and in which the method is then further intended to determine the median effective concentration (EC50) of the substance for the induction of mast cell degranulation.

9. The method according to claim 1, further comprising a step ie) of determining the agonist potential of the substance with respect to the MRGPRX2 (Mas-related G protein-coupled receptor X2).

10. The method according to claim 9, wherein step ii) of determining the inflammatory potential of the substance also makes it possible to determine whether the mast cell degranulation induced by the substance results from a pseudoallergic reaction or not and in which the method is then further intended to determine whether the mast cell degranulation induced by the substance results from a pseudoallergic reaction or not.

11. The method according to claim 2, wherein step ib) of determining the inflammatory response within the skin explant is carried out by monitoring markers of inflammation chosen from the group comprising M-CSF, G-CSF, TNFSF6, IFNA2, IFNG, RANTES, MCP-3, MCP-2, CX3CL1, TNFA, TNFB, MIF, NAMPT, TRAIL and IFNA1, TNFA, MCP1, VEGF, IP-10, MDC, MIP-1B, IL-17A, IL-17C, IL-17F, TNFB, IL-27, MCP-4, MIP-1A, IL-22, IL-1B, IL-12/IL-23p40, GM-CSF, IFNG, IL-12p70, IL-23, IL-31, EOTAXIN, IL-6, IL-4, IL-13, IL-5, IL-8, IL-15, BETA-HEXOSAMINIDASE, HISTAMINE, TRYPTASE, and CHYMASE.

12. The method according to claim 11, wherein step ic) of determining the level of mast cell degranulation within the skin explant utilizes avidin.

13. The method according to claim 12, wherein step ic) of determining the level of degranulation of the mast cells within the skin explant is carried out within a maximum period of 6 hours following the step ia) of administration.

14. The method according to claim 13, wherein step ii) of determining the inflammatory potential of the substance, and more specifically its allergic or pseudoallergic potential, is performed with regard to the proportions of mast cells undergoing low, moderate, or high levels of degranulation at the end of step ia), with:
a substance associated with a proportion of more than 50% of granulocytes exhibiting a low level of degranulation and/or less than 10% exhibiting a high level of degranulation presents a low, or even zero, allergic or pseudoallergic potential.
a substance associated with a proportion of more than 50% of granulocytes exhibiting a high level of degranulation presents a high allergic or pseudoallergic potential.

15. The method according to claim 14, further comprising a step id) of determining the level of degranulation in a culture of mast cells after incubation thereof in the presence of different concentrations of the substance.

16. The method according to claim 15, wherein step ii) of determining the inflammatory potential of the substance also makes it possible to determine the median effective concentration (EC50) of the substance for the induction of mast cell degranulation and in which the method is then further intended to determine the median effective concentration (EC50) of the substance for the induction of mast cell degranulation.

17. The method according to claim 16, further comprising a step ie) of determining the agonist potential of the substance with respect to the MRGPRX2 (Mas-related G protein-coupled receptor X2).

18. The method according to claim 2, wherein step ic) of determining the level of mast cell degranulation within the skin explant utilizes avidin.

19. The method according to claim 2, wherein step ic) of determining the level of degranulation of the mast cells within the skin explant is carried out within a maximum period of 6 hours following the step ia) of administration.

20. The method according to claim 2, wherein step ii) of determining the inflammatory potential of the substance, and more specifically its allergic or pseudoallergic potential, is performed with regard to the proportions of mast cells undergoing low, moderate, or high levels of degranulation at the end of step ia), with:
a substance associated with a proportion of more than 50% of granulocytes exhibiting a low level of degranulation and/or less than 10% exhibiting a high level of degranulation presents a low, or even zero, allergic or pseudoallergic potential.
a substance associated with a proportion of more than 50% of granulocytes exhibiting a high level of degranulation presents a high allergic or pseudoallergic potential.
